(19) United States
(12) Patent Application Publication
(10) Pub. No.: US 2021/0153617 A1
(43) Pub. Date: May 27, 2021

(54) CONTACT LENS STORAGE AND CLEANSING CONTAINER

(71) Applicant: Corneliu Ioan Doniga, Mainz (DE)

(72) Inventor: Corneliu Ioan Doniga, Mainz (DE)

(21) Appl. No.: 17/048,224

(22) PCT Filed: Apr. 18, 2019
(86) PCT No.: PCT/EP2019/060105

§ 371 Date: Oct. 16, 2020

(30) Foreign Application Priority Data
Apr. 19, 2018 (EP) 18168315.2

(51) Int. Cl. A45C 11/00; A45C 15/00; B08B 3/08; B08B 3/04; B08B 13/00; A61L 12/08

(52) U.S. Cl. CPC A45C 11/005; A45C 15/00; B08B 3/08; B08B 2203/027; B08B 13/00; A61L 12/086; B08B 2203/0211; B08B 3/044

(57) ABSTRACT

A contact lens storage and cleansing container including a number of closable contact lens chambers to which contact lens fluid can be fed from a fluid reservoir by means of a pump device via a channel system, and wherein the contact lens storage and cleansing container is suitable for a particularly long storage stability for the fluid being stored in the reservoir, The fluid reservoir can be connected, at the fluid end, by the channel system to an outlet opening via the contact lens chamber or contact lens chambers.

### SUMMARY

A contact lens storage and cleaning container (1) with a number of closable contact lens chambers (16), to which contact lens fluid can be supplied from a fluid reservoir (4) by means of a pumping device (22) via a channel system (34), should be suitable for a particularly long shelf life of the fluid stored in the reservoir (4). According to the invention, the liquid reservoir (4) flow of the liquid can be connected to an outlet opening (36) via the channel system (34) via the contact lens chamber (16) or the contact lens chambers (16).

### DESCRIPTION

The invention relates to a contact lens storage and cleaning container with a number of sealable contact lens chambers to which contact lens fluid can be supplied from a fluid reservoir by means of a pumping device via a channel system.

Contact lenses are a visual aid used worldwide. They replace glasses, which are usually not so aesthetic, by lying directly against the cornea, separated from it only by a thin tear film. There are different types of contact lenses, especially daily, monthly and annual contact lenses. Daily contact lenses should only be worn for one day and then disposed of, whereas monthly contact lenses can be used for 30 days and annual contact lenses for up to one year.

Usually, contact lenses need to be regularly cared for and kept clean, especially when used regularly. To do this, contact lenses usually for temporary storage, for example at night, and care are placed in dedicated contact lens cases and therein into a storage and cleaning liquid. Placing the lens in the storage and cleaning liquid serves on the one hand to disinfect and clean the contact lens, but is also intended to prevent the lens from drying out.

Such a treatment of the contact lenses is comparatively costly for the user, especially since usually both a storage and cleaning container for the contact lenses and a liquid dispenser for the care liquid must be kept ready. In addition, the constant transfer of solution from the solution dispenser into the lens chambers is associated with a certain unintentional loss of solution due to the small size of these chambers and is annoying in the long run.

In order to remedy these problems, from EP 2095735 B1 a contact lens storage and cleaning container of the above-mentioned type is known, in which the contact lens chambers for temporary storage of the lenses and the reservoir for storing the care liquid for at least a few days are arranged in a single, common body or housing in an integrated design. This design enables very easy handling and safe storage of the contact lenses, while strict separation of the media also reliably prevents mixing of the fresh, unused liquid with air or other media potentially contaminated with germs. To introduce the care liquid into the contact lens chambers, these are connected to the reservoir for the contact lens liquid via a channel system. The contact lens fluid can be fed from the reservoir into the contact lens chambers by means of a pumping device.

It is an objective of the present invention to provide a contact lens storage and cleaning container of the type described, which enables the liquid for the care of the contact lenses to be provided for a particularly long period of time, for example a few weeks, without the need for refilling.

According to the invention, this task is solved by connecting the liquid reservoir for the liquid flow via the channel system to an outlet opening on the contact lens chamber or chambers.

Advantageous designs of the invention are the subject of the sub-claims.

The invention is based on the consideration that an essential cause for a time limitation of the storage and provision of the care liquid in a container of the mentioned type should be seen in a risk of germination of the stored liquid. In order to counteract this risk and thus enable stockpiling for a particularly long period of time, the possibility of bacterial entry into the liquid reservoir should be consistently minimized. In a container constructed in the manner described above, the liquid reservoir is integrated into the inner body or cartridge of the container, so that contact with ambient air or other sources of germs is already avoided to a large degree. However, the liquid channel connecting the reservoir to the contact lens chambers may potentially and occasionally establish a connection with the outside environment.

In order to consistently rule out the potential entry of germs via this connection line as well, a flushing gradient should be ensured, via which the flow of media is established from the reservoir to the outside area. This is achieved by setting up a cascade-like series connection of reservoir, contact lens chambers and an outlet or disposal opening for used care liquid via the channel system. In order to dispose of used fluid in the lens chambers, the lens chambers can then be refilled with unused fluid from the reservoir via this setting, while at the same time the old, used fluid from the lens chambers is pushed out via the outlet opening. A backflow from the outside into the reservoir, which could lead to an undesired germ entry, is thus largely excluded.

The outlet opening can be arranged in the manner of a “real” outlet on an outer surface or side of the container, so that the used liquid can be pushed out via the outlet opening and directly fed to a disposal system. Alternatively, the outlet opening can also be located inside the container and lead to a separate, integrated collection reservoir for the used liquid. In this version, which no longer requires an external outlet, the disposal can then be carried out, for example, by replacing the complete collection reservoir containing the used liquid.

In a particularly preferred version, the reservoir may be of the “double chamber” type, preferably double-walled, one chamber being designed to hold fresh, unused lens solution and the other chamber to hold the used solution temporarily or permanently. The outlet opening does not open out into the outside of the overall system but into the receiving chamber, so that a discharge of the used liquid to the outside world can be avoided. In the preferred double-walled design, the receiving chamber, which is intended for the intermediate (or final) collection of the spent liquid, is located outside the actual reservoir chamber and surrounds it.

In an advantageous configuration of the invention, a bellows pump or a membrane pump is provided as pumping device. This ensures a reliable pumping function in a particularly simple and also cost-effective way. In an alternative advantageous design, a piston pump is provided as a pumping device.

In a particularly advantageous design, which is considered to be independently inventive, the container has a cartridge arranged in an outer housing in which the liquid reservoir is integrated. This design makes it particularly easy to refill after the liquid in the reservoir has been used up. The complete cartridge can be replaced. The risk of microbial contamination of the liquid during replacement is virtually eliminated, as there is no connection between the inside of the reservoir and the outside environment at any time during the replacement process. Especially in combination with the rinsing gradient resulting from the cascade-like connection of the contact lens chambers and outlet opening one behind the other, a complete rinsing process from the inside to the outside can be carried out after such an exchange by applying a small amount of the newly added care liquid, so that any germs that may be present are rinsed out and kept away from the liquid reservoir. During replacement, the existing, “used” cartridge can thus be removed from the housing and replaced with a new cartridge; the filling volume inside the cartridge for the care liquid is about 100 ml in another advantageous embodiment.

In one version of the invention, the channel system can also be integrated into the cartridge. In an alternative practical design, however, the channel system is also arranged in a separate housing part, which may be designed to be connectable to the cartridge, for example, by means of a number of plug connections.

In an alternative which is also regarded as advantageous and independently inventive, a bag with a soft, deformable outer skin can be provided instead of such a cartridge with a hard outer shell, while retaining the other design features. This design also allows the system to be filled with cleaning fluid in a particularly simple and safely sterile way by replacing the bag. In this version, too, the bag is preferably placed inside a preferably hard outer housing and protected by this.

In an additional advantageous embodiment, the or each contact lens chamber is each formed by a chamber base which can be closed with an associated chamber lid, whereby the chamber lid can be designed flexibly, particularly in the manner of a lid membrane, so that it changes its shape under pressure. The chamber base can be formed by a recess in the surface of the cartridge, whereby the respective chamber lid is arranged on the outer housing. By opening the outer housing, the chamber lids for all contact lens chambers are thus lifted and thus opened so that all chambers are equally accessible. Alternatively, the individual chamber lids can also be designed separately and independently of each other so that they can be opened independently of each other.

However, the chamber lid can also be located between the cartridge and the “system”, in which case the system advantageously includes at least one pump modality (=possibility to operate the pump, not necessarily the pump itself). The actual pump can be actuated, for example, through an inner slotted hole or through lateral devices.

Advantageously, the or each chamber cover is designed as a deformable membrane. This design makes it particularly easy to reliably seal the lens chambers when closed. In particular, the membrane can be designed in such a way that it forms a sealing lip at its circumferential edge which makes contact with the cartridge surface. As soon as pressure is then created in the lens chamber during filling with liquid, the membrane is moved upwards towards the outer housing, creating space for the inflowing liquid. At the same time, pressure is exerted on the lateral sealing lip so that contact with the “counterpart”, i.e. the surface of the cartridge, is intensified and the sealing effect is enhanced.

In particularly advantageous embodiment, the design of the chamber lid as a deformable membrane can also be used to provide an indicator function that enables the user to determine in a particularly simple way whether the respective lens chamber is currently filled with care fluid. For this purpose, the deformable membrane preferably interacts with an indicator element that can be seen from outside the outer housing, for example with an indicator pin attached to its surface, which is guided through an associated hole in the outer housing and allows the user to determine in a simple manner whether the membrane is currently deformed up to close to the outer housing. Alternatively, such an indication function could also be provided via an electronic display of the filling level, for example by means of an LED.

In its advantageous design, the cartridge is a comparatively complex component with a number of integrated functions to make it particularly easy to handle. In order to use this in a particularly effective way, the cartridge is advantageously designed as a reusable product which can be refilled for a new use cycle after the liquid in the reservoir has been used up. It is also advantageous that the liquid reservoir of the container can be refilled.

A particularly high level of functionality and good usability can be achieved by providing an appropriately designed closure and locking mechanism. For this purpose, a rotary closure is provided for opening or closing the lid of the outer housing, which is particularly advantageous for this purpose. The release valves of the channel system and thus the media-side connection within the channel system can also be controlled via this rotary closure. As an example, a closure designed in this way can offer the following setting options:

If the cap is pulled out slightly (it will then preferably snap into place), the liquid is released from the lens chambers to the outside, e.g. for disposal or rinsing. If, on the other hand, the cap is turned in one direction, e.g. to the right, the lid opens; however, the cartridge is blocked in the housing and cannot be removed. If, on the other hand, the rotary closure is turned 180° in the opposite direction, for example to the left (this should preferably only be possible with the lid open), the cartridge can be easily removed and replaced with a new, full cartridge. When the cap is fully open (e.g. pulled out) and the pump button is pressed, the lenses in the chambers are rinsed and the liquid is simultaneously let out and disposed of. In this mode, liquid can also be dispensed into the user's hand in a targeted manner, e.g. to rub his lenses manually.

In a particularly preferred design, the container, especially its cartridge, is also equipped to provide a wide range of additional functions designed to provide the user with improved usability and/or even greater ease of use. Such additional functions are preferably provided electronically, and for this purpose, the container, preferably its cartridge, includes an energy storage device for electrical energy. In particular, a battery or accumulator can be provided as energy storage.

As a particularly preferred additional function, a mechanical cleaning function for the contact lenses can be provided here in addition to treatment in the care liquid, if the lenses are located in the lens chambers. This design allows the lenses to be subjected to a combined mechanical and chemical treatment, so that the efficacy of these treatments complement each other synergistically. For this purpose, the energy storage device is advantageously connected to a number of vibrating elements associated with the chamber lid(s). Especially in combination with the membrane lids preferably provided for the lens chambers, the lids can thus be designed as “vibration membranes”. If these vibrate when the lens is inserted (similar to vibrating toothbrushes), reliable cleaning of the lenses can take place even in the presence of a small amount of lens fluid. The contaminated liquid from the lens chambers after treatment can be disposed of by pumping and then pulling out the rotary shutter. This can also be done in an electric version, preferably in fully automatic mode, in which the user does not have to do anything. The contaminated liquid is then automatically pumped into the second reservoir, after which the lenses are immersed in a fresh bath of liquid for storage.

Other particularly preferred additional functions that can be provided by using the energy storage system are—preferably individually or in any combination with each other—electrically driven pumps for transporting the care fluid in the channel system, an electrical display unit, for example for the level of the care fluid in the reservoir and/or in the lens chambers, remaining time until the next care treatment or similar, or the stimulation of electroluminescent properties of the contact lenses. In particularly advantageous further training courses, UV-C LEDs can also be installed which have a disinfecting effect on the lenses. Light of the type UV-A could thus be used, which is favourable for the excitation of luminescence, and/or particularly preferably also UV light with a shorter wavelength (UV-C) of, for example, about 200 nm. In addition, in particularly advantageous further training, which is regarded as independently inventive, reflective structures or elements are provided in the lens chambers, preferably on the cover membranes, which enable or favor the homogenization of light within the respective lens chamber.

The advantages achieved with the invention consist in particular in the fact that, due to the cascade-like series connection of the liquid reservoir, lens chambers and outlet or disposal opening on the media side, the media transport of the care liquid takes place consistently from the inside to the outside, i.e. from the liquid reservoir to the outlet opening, especially during the rinsing process, and a backward flow into the reservoir is minimized or even completely excluded. This prevents germs from entering the reservoir from the outside, thus significantly increasing the shelf life and thus the storage time of the liquid in the reservoir.

An example of the invention is explained in detail by means of a drawing. The figures show:

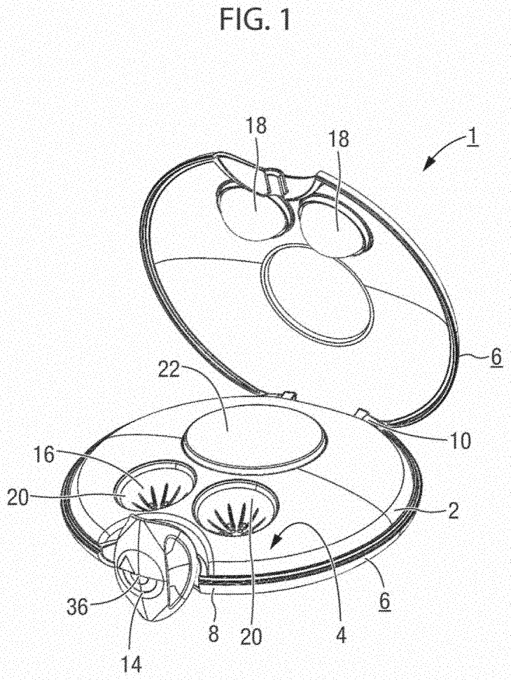

FIG. 1 a contact lens storage and cleaning container in perspective view,

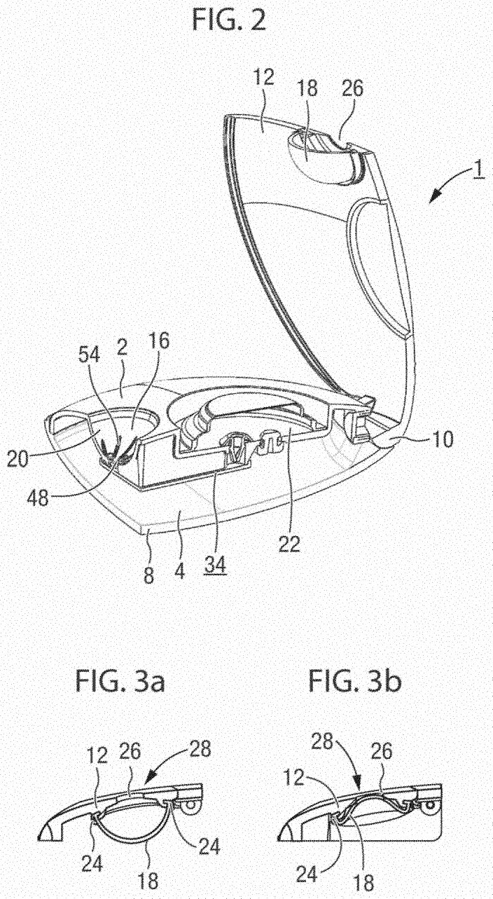

FIG. 2 the contact lens storage and cleaning container according to FIG. 1 in perspective section,

FIG. 3 an excerpt from FIG. 2,

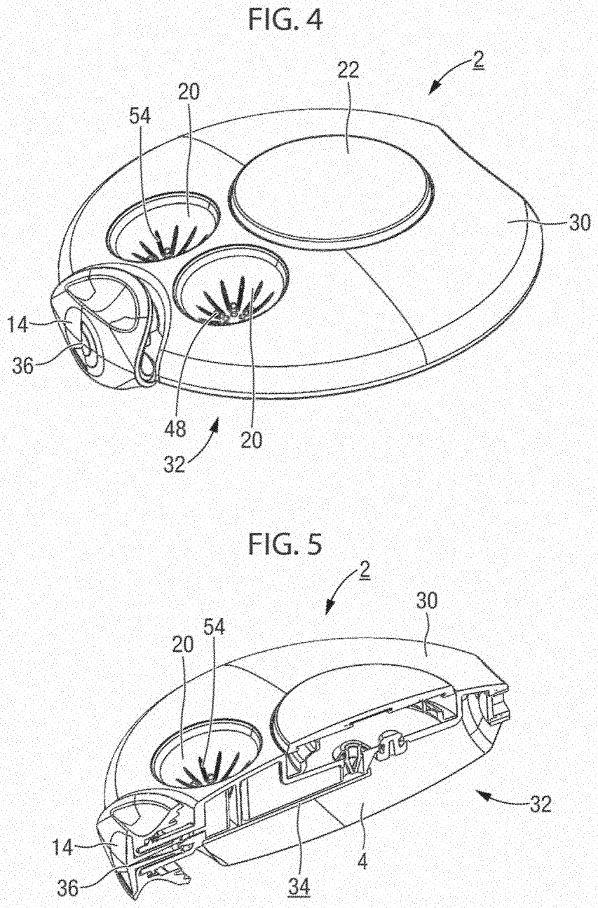

FIG. 4 a cartridge of the contact lens storage and cleaning container in perspective view,

FIG. 5 the cartouche according to FIG. 4 in perspective section,

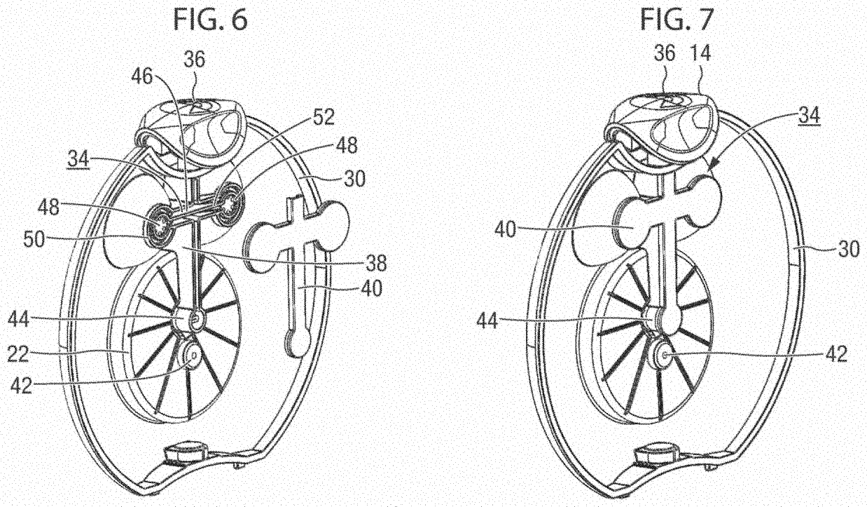

FIG. 6 the upper cartridge shell of the cartridge according to FIG. 4 in an exploded view from below,

FIG. 7 the upper cartridge shell of the cartridge according to FIG. 4 in assembled condition from below,

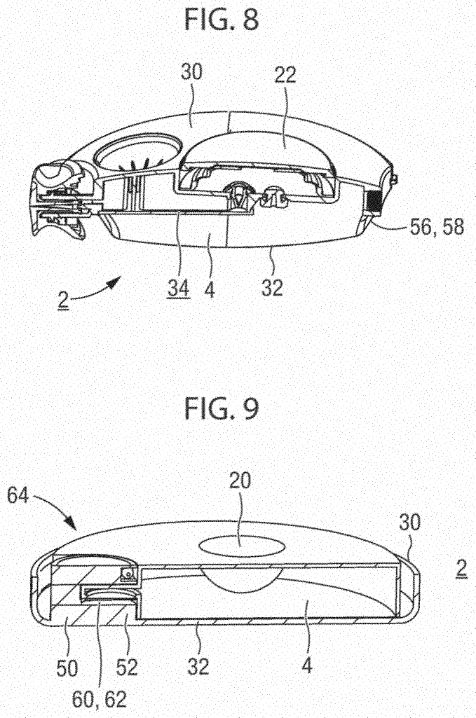

FIG. 8 an alternative view of the cartouche according to FIG. 4 in section,

FIG. 9 an alternative design of a cartridge in section,

FIG. 10, 11 one lid closure each of the container according to FIG. 1 in exploded view,

FIG. 12 an alternative design of a contact lens storage and cleaning container in perspective view,

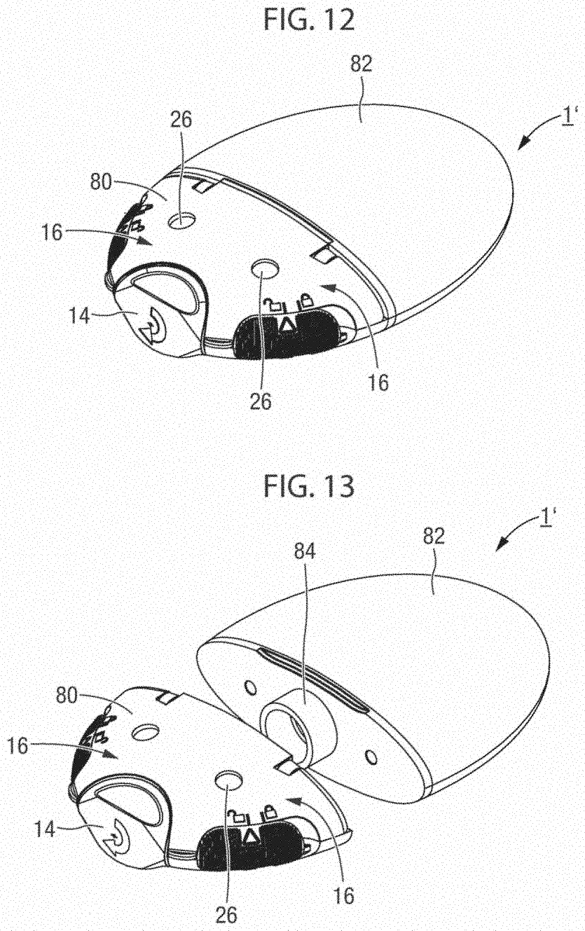

FIG. 13 a perspective exploded view of the contact lens storage and cleaning container as shown in FIG. 12,

FIG. 14 a reservoir module of the contact lens—storage and cleaning container according to FIG. 12 perspectively in exploded view,

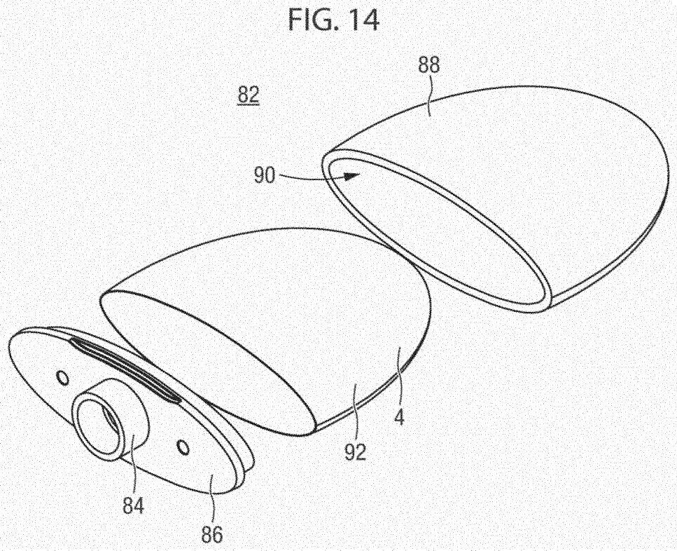

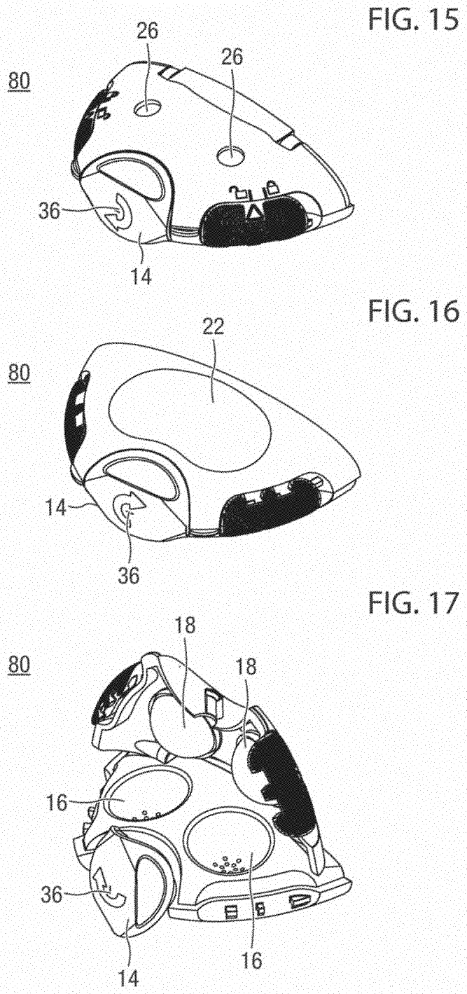

FIGS. 15-17 one basic module of each contact lens in perspective—storage and cleaning container according to FIG. 12,

FIG. 18 the basic module according to FIG. 15 with removed lens chambers, and

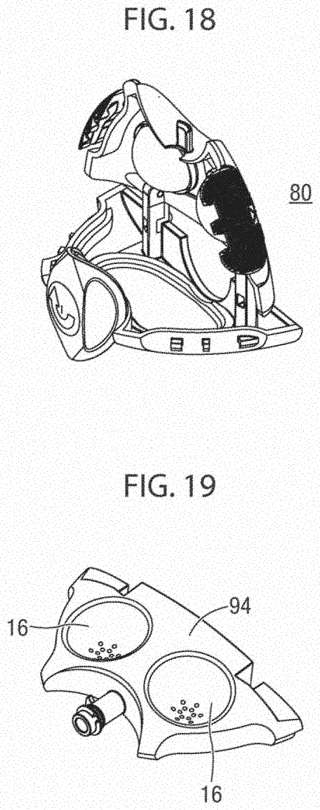

FIG. 19 the lens chambers of the basic module according to FIG. 15,

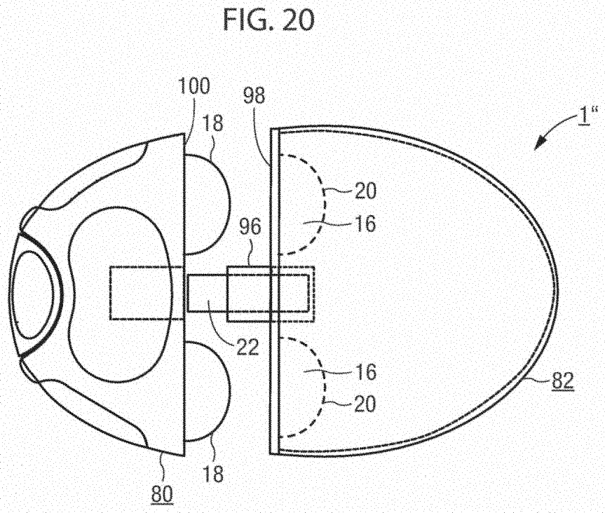

FIG. 20 another alternative design of a contact lens storage and cleaning container in longitudinal section,

FIG. 21 the contact lens storage and cleaning container according to FIG. 20 in open position in top view,

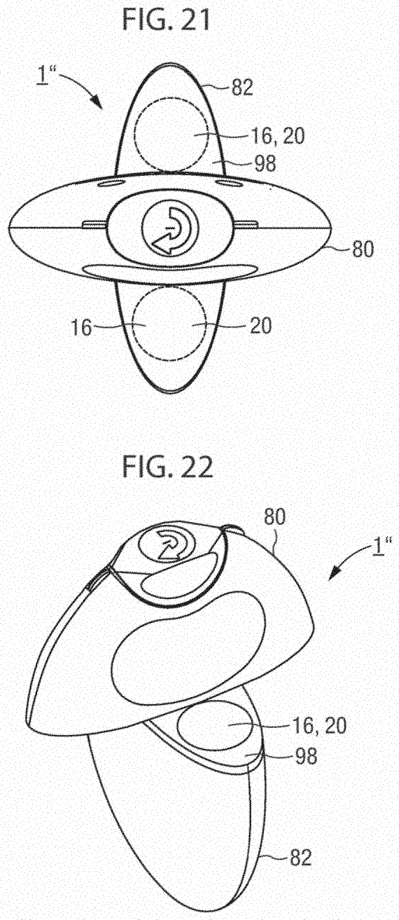

FIG. 22 the contact lens storage and cleaning container as shown in FIG. 20 in open position in perspective view,

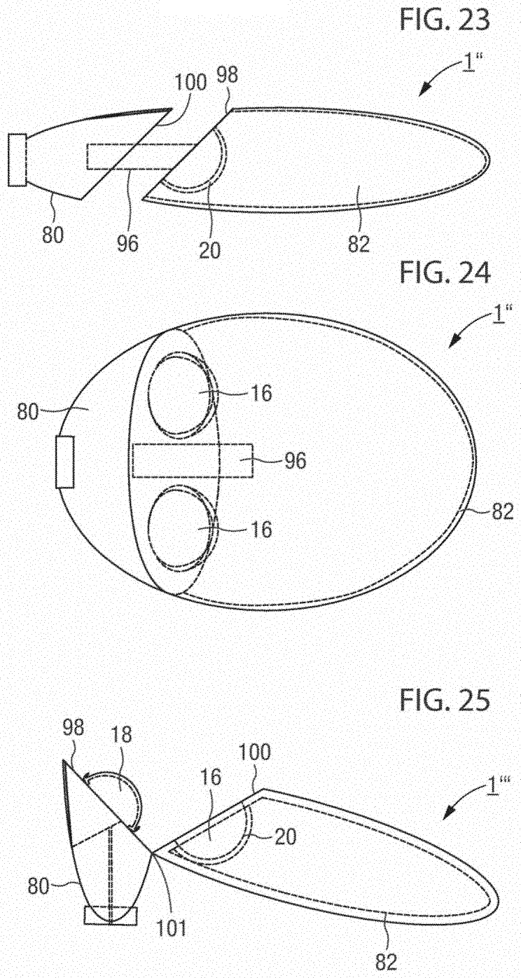

FIG. 23 the contact lens storage and cleaning container as shown in FIG. 20 with bevelled end faces in open position in side view,

FIG. 24 the contact lens storage and cleaning container as shown in FIG. 20 with bevelled end faces in the open position as seen from below,

FIG. 25 an alternative contact lens storage and cleaning container with bevelled end faces in open position in side view,

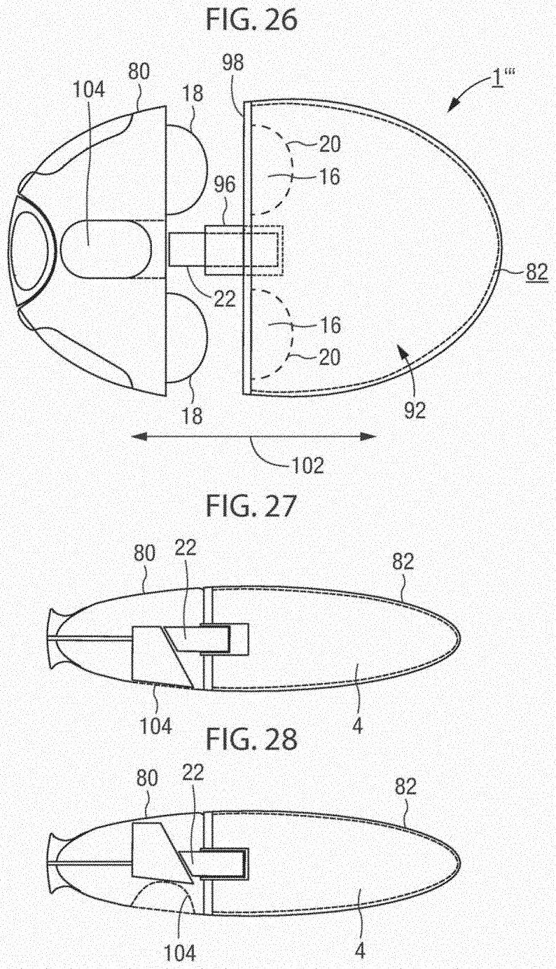

FIG. 26 another alternative version of a contact lens storage and cleaning container in bottom view,

FIG. 27 another alternative version of a contact lens storage and cleaning container with integrated pump in longitudinal section,

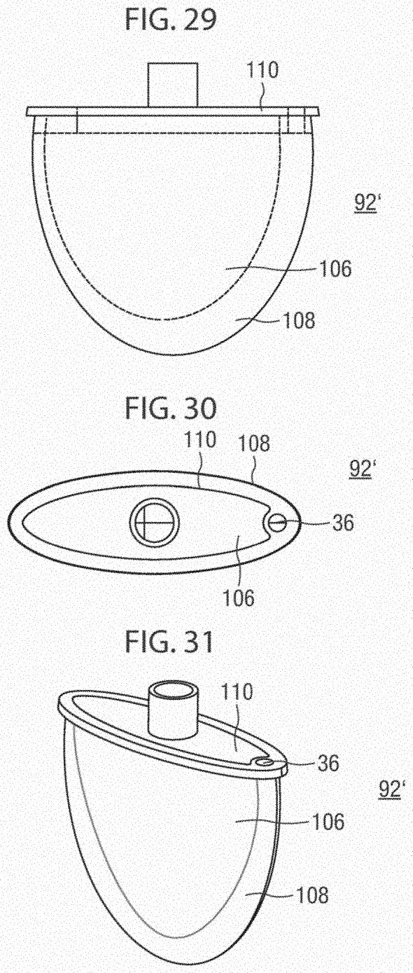

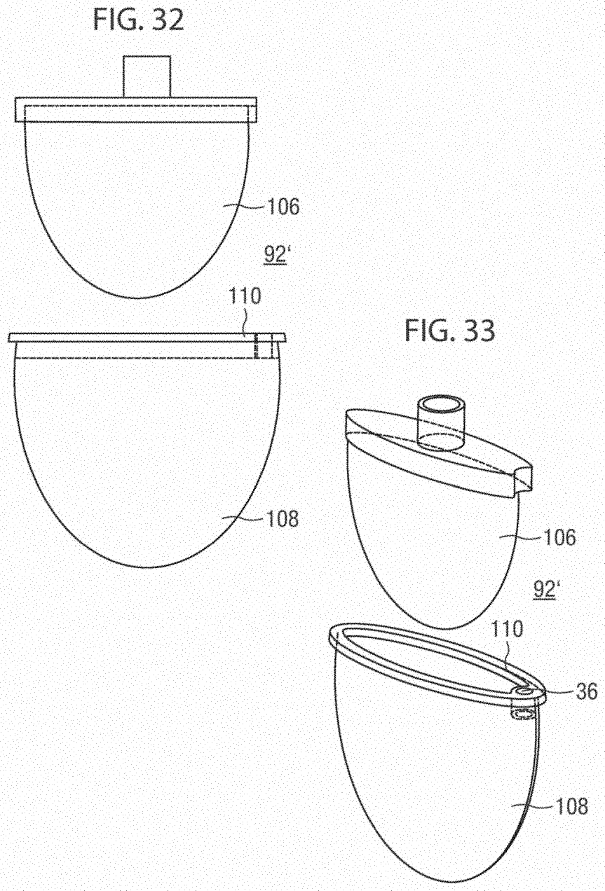

FIG. 28 the tank according to FIG. 27 in longitudinal section with actuated pump, FIG. 29 an alternative design of a reservoir bag on average, FIG. 30 the reservoir bag according to FIG. 29 in top view, FIG. 31 the reservoir bag according to FIG. 29 in perspective view, FIG. 32 the reservoir bag according to FIG. 29 in exploded view in side view, and

FIG. 33 the reservoir bag according to FIG. 29 in exploded view in perspective.

Identical parts are marked with the same reference signs in all figures.

The contact lens storage and cleaning container 1, hereafter referred to simply as “container 1”, as shown in FIGS. 1, 2, is used for the temporary storage of contact lenses and a supply of solution for cleaning and care of the lenses. For this purpose, container 1 comprises a reservoir 4 integrated into an interchangeable cartridge 2 or a tank with a capacity of approximately 80 to 100 ml, preferably and as in the example, in which the cleaning or care fluid can be stored. The cartridge 2 is detachably mounted in an outer housing 6. In the design example, the outer housing 6 is formed by a lower base 8 and by a housing cover 12 hinged to it by a hinge 10. In the closed position, the housing cover 12 can be locked to the base 8 by means of a rotatably designed cover lock 14.

For storing the contact lenses, container 1 has a number of contact lens chambers 16, in the design example two, and in the design example two. The contact lens chambers 16, which in the preferred design example each have an internal volume and thus a capacity of about 1.5 ml, are each formed by a chamber lid 18 and by an associated chamber base 20, whereby the chamber base 20 can be closed in the closed state by its associated chamber lid 18. The chamber base 20 is formed by a recess in the surface of the cartridge 2, whereby the associated chamber lid 18 is located on the housing cover 12 of the outer housing 6. By opening or closing the outer housing 6, the contact lens chambers 16 are thus opened or closed equally and simultaneously.

When storing the contact lenses in the contact lens chambers 16, it is intended that these are filled with care liquid from the reservoir 4, so that the contact lenses are kept moist and, if necessary, cleaned and/or disinfected. For this purpose, a pumping device 22 is integrated in the cartridge 2, which can be operated manually in the manner of a pressure pump and is designed as a bellows in the design example, by means of which liquid can be pumped from the reservoir 4 into the contact lens chambers 16.

As can be seen in particular from the enlarged sections of the illustrations in FIGS. 3a,b, each chamber cover 18 is designed as a deformable membrane. The membrane forms a sealing lip 24 on its circumferential edge which makes contact with the cartridge surface. FIG. 3a shows the membrane in a relaxed resting state. When the respective contact lens chamber 16 is filled with liquid, pressure builds up in the lens chamber accordingly, so that the membrane yields upwards towards the outer housing 6 and frees space for the inflowing liquid. This condition is shown in FIG. 3b. At the same time, pressure is exerted on the lateral sealing lip 24, so that contact with the surface of the cartridge 2 and thus the sealing effect is increased. The chamber lids 18 are attached to the outer housing 6 by means of a carrier ring with undercut, which is located on the outer housing 6 and onto which the respective member can be easily fitted. The two diaphragms forming the chamber lids 18 are of identical and rotationally symmetrical design, so that the user does not have to pay any significant attention to alignment or chamber side when replacing them.

In the preferred embodiment shown, the deformability of the membrane is also used to provide an indicator function that enables the user to determine whether the respective contact lens chamber is currently filled with care fluid. For this purpose, a viewing window 26 or an opening is arranged in the housing cover 12 above the respective membrane. Through the window 26, the user can see from the outside whether the membrane underneath is deflected upwards and whether the contact lens chamber 16 is filled accordingly. This visibility can be enhanced, for example, by a suitable marker on the top of the membrane and/or an indicator pin on its surface. These elements including the viewing window 26 thus form an indicator element 28 for the filling status of the respective contact lens chamber 16, which can be viewed from outside the outer housing 6.

Cartridge 2, which is arranged in the outer casing 6, shown in FIG. 4 in perspective in side view and in FIG. 5 in perspective in sectional view, and whose design is considered to be independently inventive, comprises an upper cartridge shell 30 and a lower cartridge shell 32, which are welded together at their circumferential edge to form the cartridge casing. The cartridge shell 30 is preferably produced as an injection moulded part made of polypropylene (PP) or polyethylene (PE). In contrast, the cartridge shell 32 is designed as a comparatively elastic component so that it can contract. The preferred material for this is TPE, but silicone or an expandable LDPE is also conceivable. However, these parts are not produced by injection moulding. Alternatively, a rigid ring could be provided, which should be injection moulded and could consist of an elastic material in the middle. This could facilitate the welding process. The integrated elements of cartridge 2 are the liquid reservoir 4 and the pumping device 22, and the lid closure 14 is also attached to cartridge 2. Cartridge 2 also incorporates a channel system 34 that allows the liquid to be pumped from reservoir 4 to the contact lens chambers 16 by means of pumping device 22.

Cartridge 2 and, with it, container 1 are specifically designed to ensure a particu larly long shelf life for the cleaning or care liquid in reservoir 4. Special attention has been paid to the fact that the shelf life of the liquid could be limited by penetrating germ loads. To counteract this, the channel system 34 is designed in such a way that the liquid can be fed by means of the pumping device 22 via the channel system 34 from the liquid reservoir 4 via the contact lens chambers 16 to an outlet opening 36 located in the lid closure 14. Thus, on the liquid side, a cascade-like series connection of the elements reservoir 4, contact lens chamber 16 and outlet opening 36 can be produced, so that the liquid can only flow in the direction outwards to the outlet opening 36 and a backward flow into the reservoir 4 can be minimized or almost excluded. The rinsing of the lenses in the chambers or the release of the contaminated liquid from the chambers when the lid closure is pulled out is also facilitated by the chamber lids designed as lid membranes. This is because they return to the deflected starting position as soon as there is a pressure relief, for example by opening the outer channel by pulling out the closure.

To illustrate this design, the upper cartridge shell 30 is shown in FIG. 6 (as an exploded view) and in FIG. 7 (assembled) in a view from below. As can be seen from

FIGS. 6, 7, the upper cartridge shell 30, which is designed as an injection moulded part, has a moulded-on channel piece 38 on its lower side facing the interior and thus the reservoir 4, which is connected to a welded-on channel plate 40 to form the channel system 34. As can be seen in these illustrations, the pumping device 22 is connected on the liquid side to the reservoir 4 via a shield valve 42. This shield valve 42 is to prevent overpressure in the pump chamber. In case of overpressure it opens into the reservoir: as soon as the pressure in the pump becomes too high, a “short circuit” occurs. However, as the liquid from the channel system never enters the pump chamber again, this short circuit does not lead to contamination of the reservoir liquid. Furthermore, the pumping device 22 is connected to the channel system 34 via a non-return valve 44, whereby the non-return valve 44, also in the form of a unidirectional design, only allows liquid to flow out of the interior of the pumping device 22 into the channel system 34, but not liquid to flow back from the channel system 34 into the pumping device 22.

In order to prevent excessive pressure from being generated in the contact lens chambers 16 when the pumping device 22 is operated, it is also protected by a pressure relief valve. This can be formed by the non-return valve 44.

During use, cleaning fluid is first introduced from cartridge 4 into the reservoir of pumping device 22. From there, the cleaning fluid passes through the channel system 34 into the contact lens chambers 16 and rinses and cleans the lenses located there. At the end of the cleaning process and during or before the lenses are removed from the contact lens chambers 16, the used cleaning fluid escapes through the outlet 36.

The channel system 34 comprises a centrally arranged inflow channel 46 leading into the contact lens chambers 16. This channel opens into the interior of the respective contact lens chamber 16 via a number—six in the embodiment shown—of inlet holes 48 arranged in the respective chamber floor 20. In the outlet or outflow direction, the respective contact lens chamber 16 is connected to the lid closure 14 via outlet holes 50 also arranged in the chamber floor 20 and an outlet channel 52 placed next to the inflow channel 46. In order to ensure reliable media exchange even when contact lenses are inserted into the contact lens chambers 16 and to prevent unintentional closure of the inlet and/or outlet holes 48, 50 by the contact lenses, the chamber floor 20 is provided with a number of support bars 54 on which the contact lens rests at a distance from the inlet and/or outlet holes 48, 50.

The liquid is guided—among other functions—by means of the lid closure 14, which is designed on the one hand as a rotary closure for opening or closing the housing lid 12 of the outer housing 6. In addition, the lid lock 14 also controls the integrated release valves of the channel system 34 (not shown in detail) and thus the media-side connection within the channel system 34. The following setting options are provided in the design example, although other specific configurations are of course also possible:

When the lid catch 14 is pulled out slightly (and preferably clicks into place), the outlet opening 36 is released and thus the liquid from the contact lens chambers 16 is discharged to the outside, e.g. for disposal or rinsing. If, on the other hand, the lid closure 14 is turned in one direction, e.g. to the right, the housing lid 12 opens; the cartridge 2, however, is blocked in the outer housing 6 and cannot be removed.

The cartridge is blocked in the housing by the closure as long as the latter is not turned 180° to the left (with the lid open). However, the rotation of the cap is irrelevant for liquid dispensing. However, the user can only pull the cap out at the beginning; rotation is only possible when the cap is pulled out.

If, on the other hand, the lid closure 14 is turned 180° in the opposite direction, for example to the left (this should preferably only be possible with the lid open), cartridge 2 can be easily removed and replaced by a new, full cartridge 2. When the lid closure 14 is fully open (e.g. pulled out) and the button of the pumping device 22 is pressed, the lenses in the chambers are rinsed and the liquid is simultaneously let out and disposed of. In this mode, it is also possible to pour liquid into the user's hand in a targeted manner, e.g. to rub his lenses manually.

As can be seen from the alternative view of cartridge 2 in FIG. 8, reservoir 4 is designed to be refillable. For this purpose, a number of filling openings 56 are provided at the end of the cartridge body on the side opposite the outlet opening 36 and the twist cap 14, through which the liquid can be introduced into the reservoir in the manner of a refuelling using a suitably designed filling lance. Each filling opening 56 is assigned a sealing plug 58, with which the respective opening can be closed after filling. In principle, a single filling opening 56 could be provided, through which cartridge 2 can be refilled several times, in the manner of a reusable product, and in itself an unlimited number of times. Alternatively, however, the systems consisting of filling opening 56 and associated stopper 58 can also be designed for single use only, so that the number of possible refilling processes is limited by the number of “unused” filling openings 56 still available.

In a particularly preferred version, which is considered to be independently inventive, cartridge 2 and with it container 1 can also be equipped with electrically activatable or controlled units for providing further additional functions. In a design example suitable for this purpose, as shown in the sectional view of cartridge 2 in FIG. 9, this includes an energy storage unit 60 for electrical energy. This can be designed as a battery, but in the preferred design example shown, an accumulator 62 is provided as energy storage 60, which can be connected to an external power supply via a charging socket 64 integrated in cartridge 2 for the purpose of recharging and can be recharged via this socket. In particular, there may be a possibility of recharging via an induction coil, either integrated or external.

The use of the energy storage 60 can be provided for a variety of possible applications. As a particularly preferred additional function, a mechanical cleaning function for the contact lenses can be provided in addition to the treatment in the care liquid if the lenses are in the contact lens chambers 16. This design allows the lenses to be subjected to a combined mechanical and chemical treatment, so that the efficacy of these treatments complement each other synergistically. For this purpose, the energy accumulator 60 can be connected to a number of vibration elements associated with the chamber lids 18. The chamber lids 18 of the contact lens chambers 16, which are designed as membrane lids, are accordingly designed as “vibrating membranes”. If these vibrate when the lens is inserted, reliable cleaning of the lenses can take place even in the presence of a small amount of lens fluid.

Other additional functions that can be provided by using the energy storage 60 are, for example, electrically driven pumps to transport the care fluid in the channel system 34, an electrical display unit, for example, for the level of the care fluid in reservoir 4 and/or in the lens chambers, remaining time until the next care treatment or similar, or the stimulation of electroluminescent properties of the contact lenses, e.g. by generating electromagnetic waves (in the case of UV light).

An example of this is a pump that can be driven by the energy accumulator 60 and is integrated in cartridge 2.

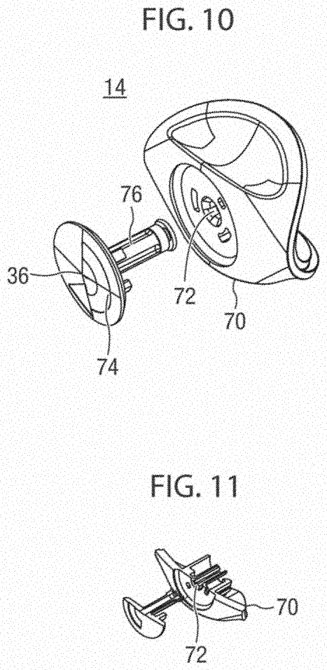

The cover fastener 14 is in the particularly preferred embodiment, as shown in the exploded views in FIGS. 10 and 11, itself designed in several parts. As components, it comprises the outer actual control panel 70, which is made of a comparatively hard plastic material and has suitable shapes as grip surfaces for operation. A valve part 74 made of a comparatively soft plastic material can be inserted into a mounting channel 72 in the central area of the operating unit 70. This has a channel piece 76, which can be connected on the inlet side to the channel system 34 in the cartridge 2 and forms the outlet opening 36 on the outlet side. The channel piece 76, which is designed in the manner of a soft component, is shaped similarly to a beak valve, so that no more liquid drips from the inside of the container after the rinsing process is completed.

An alternative design of a contact lens storage and cleaning container 1′ according to the invention is shown in FIGS. 12 ff. In this variant, the contact lens storage and cleaning container 1′ essentially comprises two modules, namely a basic module 80 on the one hand and a reservoir module 82 on the other. As can be seen from the general view in FIG. 12 and the exploded view in FIG. 13, the modules 80, 82 can be plugged together. The basic module 80 shown in FIGS. 12, 13 with closed contact lens chambers 16 comprises the essential components intended for the handling of the contact lenses in accordance with the invention, namely for example the actual contact lens chambers 16, the cover closure 14, the channel system 34, the pumping device 22 and the like. The reservoir module 82, on the other hand, is designed to accommodate reservoir 4 for the cleaning fluid. The mechanical connection between the modules 80, 82 is established by a number of connectors 84, which are moulded onto one of the modules 80, 82 and can be inserted into the corresponding sockets in the other module 80, 82. The connecting plug(s) 84 can be equipped with integrated connecting channels for connecting reservoir 4 for the cleaning fluid with the channel system 34 in the base module 80, so that in addition to the mechanical connection of the modules 80, 82 with each other, the fluid-side connection of reservoir 4 with the channel system 34 is also realized via the connecting plugs 84.

The reservoir module 82 shown in the exploded view in FIG. 14 is in turn constructed in several parts and comprises a front panel 86 provided for the connection with the base module 80 and equipped with the connecting plugs 84 as well as a housing shell 88 which can be connected to the front panel to form an outer housing. Reservoir 4 for the cleaning liquid can be inserted into the inner volume 90 formed by the front panel 86 and housing shell 88 in the assembled state. The reservoir can be formed by the cartridge 2 according to the above mentioned design; alternatively and in a design considered as independently inventive, however, a reservoir bag 92 with a soft, deformable outer skin is provided in the example shown to form the reservoir 4. This design allows a particularly easy replacement of the reservoir 4, for the replacement of the cleaning fluid, with a particularly high flexibility of the absorption quantity of the reservoir 4. The comparatively stable housing shell 88 protects the reservoir bag 92, which is designed with a soft outer skin, against mechanical damage.

The basic module 80 shown in FIG. 15 in perspective upper view, in FIG. 16 in perspective lower view and in FIG. 17 in the condition with opened contact lens chambers 16 comprises as shown above the components contact lens chambers 16, pump device 22, lid closure 14 as well as the channel system 34.

The container according to this design example is thus now divided into two parts, the front part, which is provided as the base module 80 and contains the contact lens chambers 16, the rotary closure 14, the expanding lid membranes with the associated lid, the lower shell (to accommodate the lens chambers 16), the pump 22 and the valves, and possibly additional locking mechanisms (shown as “slides” in the pictures).

The rear part, designed as a reservoir module 82, in this embodiment is only intended for cartridge 4, which can be produced extremely cheaply in this way. Cartridge 4 essentially consists of a soft bag welded to a hard component, which has a connection called a “connector” that can be tightly connected to the front part (e.g. by screwing or plugging it in). In this way, the connection between the cartridge and the front part is made.

As with an infusion bag, the bag itself contracts as soon as liquid is taken out of it. The bag is covered by a hard shell, which can be produced to the same high quality as the front part. In this case, the cartridge is replaced by removing the empty bag from the cartridge tray and replacing it with a full bag. This bag forms a unit with the hard component above it.

The contact lens chambers 16 or their lower half of the housing are formed by an appropriately designed moulded part 92, which is inserted into the base module 80 and can be removed from it. In FIG. 18, the front part or base module 80 of the container is shown in a state where the reservoir module 82 is uncoupled and the moulded part 94 has been removed. In this case, the base module 80 forms an empty tray. The removed moulding 94 is shown in FIG. 19.

Another alternative design of a contact lens storage and cleaning container 1″ according to the invention is shown in FIGS. 20 ff. In this variant, a division into essentially two modules is also provided, namely the base module 80 on the one hand and the reservoir module 82 on the other hand. In this variant, the base module 80 is designed in the manner of a rotatable head part and is designed to be rotatable relative to the reservoir module 82 in a version considered to be inde-pendently inventive. For this purpose, the base part 80 is connected to the reservoir part 82 via a connecting flange 96, as shown in the sectional drawing in FIG. 20. The connecting flange 96 is provided with an integrated channel for exchanging the lens fluid between reservoir module 82 and base part 80. The pump unit 22, which is designed as a piston pump in this design example, is also integrated into this channel. The base part 80 is arranged on the connecting flange 96 so that it can be moved in the longitudinal direction and can be operated in principle in a similar way to the cover lock 14: when the base part 80 is “pulled out”, i.e. moved away from the reservoir module 82, it comes into a position on the connecting flange 96 in which it can be rotated on the latter in the manner of a shaft bearing. The contact lens chambers 16 are formed in this independently inventive design in the connection area of the two modules by their end faces 98,100, whereby the chamber floor 20 of each lens chamber 16 is integrated into the end face 98 of the reservoir module 82 in the manner of a moulding and the chamber cover 18, each designed as a membrane, is attached to the end face 100 of the base module 80. The lens chambers 16 are thus closed by moving the base module 80 with its front face 100 on the connecting flange 96 up to the front face 98 of the reservoir module 82.

In order to be able to remove the lenses from the lens chambers 16, the base module 80 in this design must therefore be moved away from the reservoir module 82 on the connecting flange 96 and then rotated on the connecting flange 96 by an opening angle, in the design example and particularly preferred 90°. This open position for removing the lenses is shown in the views according to FIG. 21 (top view) and FIG. 22 (perspective view).

This alternative version of the contact lens storage and cleaning container 1″ with bevelled front faces 98, 100 of base module 80 and reservoir module 82 is designed in a particularly advantageous further training as shown with opened lens chambers 16 in side view in FIG. 23 and in bottom view in FIG. 24. This particularly preferred design has the essential advantage that the access area of the lens chambers 16 is enlarged and thus the removal of the lenses is easier. In addition, the volume of the lens chambers 16 is comparatively enlarged, so that they can be integrated into the lens chambers 98, 100 even if the end faces 98, 100 are kept comparatively narrow or slim.

As an alternative to the variant shown in FIGS. 23, 24, in which the base module 80 and the reservoir module 82 can be moved relative to each other in the longitudinal direction, whereby when pulled apart the lens chambers 16 are exposed and can thus be made accessible, it is also possible to be connected to the reservoir module 82 by means of a hinge 101 in accordance with the variant of the base module 80 shown in FIG. 25. In FIG. 25 this variant is shown in the open state, in which the lens chambers 16 are also open and the lenses inside are thus accessible.

A further alternative version of a contact lens storage and cleaning container 1′″ according to the invention is shown in FIGS. 26 ff. In this variant, too, a division into essentially two modules is provided, namely the base module 80 on the one hand and the reservoir module 82 on the other hand. In this variant, the base module 80 can be plugged onto the connecting flange 96 for connection to the reservoir module 82 or can be mounted on it in a rotatable manner. In this version, it is regarded as independently inventive that a piston pump provided as pumping device 22 is integrated into the connecting flange 96 and thus into the reservoir module 82 or the reservoir bag 92 or the cartridge 2. This ensures that if cartridge 2 cannot be refilled by the user, a fresh, unused pump is automatically provided after each cartridge change.

The pumping operation can be carried out, as symbolized by the double arrow 102 in the plan view according to FIG. 26, by actuating the pumping device 22 by pressing together and pulling apart the base and reservoir modules 80, 82 or alternatively by actuating a pump button 104 located in the housing and acting on the piston pump. A variant of the contact lens storage and cleaning container 1′″ with such a pump button 104 is shown in the longitudinal section in FIGS. 27 (uncompressed pump) and 28 (compressed pump). The pump 22 can be actuated as shown by the pump button 104 or through an oblong hole in the base module 80 or by pump handles arranged on the side of the base module. This preferred de-sign has the advantage that the user can use only one hand to put liquid into his other hand to rub the lenses. However, when pulling the base and reservoir apart or pushing them together, two hands are needed. Alternatively, instead of the pump button 104, a slotted hole can be provided to allow direct access to the pump 22.

Alternatively, and still conceivable, the pump could be actuated by turning the base module 80 against the reservoir module 82 in several or more stages, for example, provided that the lens chambers 16 remain closed during this process. Causing a pumping action in this way (e.g. turning 6 times) would then render an additional pump button obsolete. One rotation could, for example, pump 0.5 ml of liquid into the lens chambers (2×0.25 ml). The dosage would thus be very precise.

An alternative version of the 92′ reservoir bag, which is considered to be independently inventive, is shown in FIGS. 29-33. As can be seen from these illustrations, the 92′ reservoir bag 92′ in this design is made up of several parts, whereby an inner reservoir bag 106 forms the actual reservoir and is intended for storing the lens fluid. The inner reservoir bag 106 is arranged inside an outer reservoir bag 108 when assembled. This bag is originally empty, i.e. not filled, and serves as a collection container for the used lens solution after the rinsing process. For this purpose, the channel system 34 is designed in such a way that the outlet opening 36 for the used lens fluid is not placed on the outer housing 6, but is arranged in the cover plate 110 of the reservoir bag 92′ in such a way that it opens into the space formed by the reservoir bags 106, 108. This means that an external outlet is no longer necessary; the entire lens cleaning process takes place inside the storage and cleaning container.

### LIST OF REFERENCE SIGNS

1, 1′ Contact lens storage and cleaning container

2 Cartridges

4 Reservoir

6 Outer casing

8 Floor

10 Hinge

12 Housing cover

14 Cover closure

16 Contact lens chamber

18 Chamber lid

20 Chamber floor

22 Pump device

24 sealing lip

26 Viewing window

28 Display element

30 Upper cartridge tray

32 Lower cartridge tray

34 Duct system

36 Outlet opening

38 Channel piece

40 channel plate

42, 44 Check valve

46 Inflow channel

48 Inlet hole

50 Air outlet hole

52 Outflow channel

54 Supporting bar

56 Filling opening

58 Closing plugs

60 Energy storage

62 Accumulator

64 Charging socket

66 Peristaltic pump

70 Control panel

80 basic module

82 Reservoir module

84 Connector plug

86 Front panel

88 Housing shell

90 Interior volume

92, 92′ Reservoir bag

94 Moulded part

96 Connecting flange

98, 100 Face area

101 Hinge

102 double arrow

104 Pump knob

106 inner reservoir bag

108 outer reservoir bag

110 Cover plate

In the claims:

1. A contact lens storage and cleaning container with one or more closable contact lens chambers, to which contact lens liquid can be supplied from a liquid reservoir by a pump device via a channel system, wherein for liquid flow the liquid reservoir is connected to an outlet opening via the one or more contact lens chambers via the channel system.

2. Container according to claim 1, wherein the pump device is a membrane pump.

3. Container according to claim 1, further comprising a cartridge arranged in an outer housing into which the liquid reservoir is integrated.

4. Container according to claim 1, wherein the channel system is integrated into a separate housing part.

5. Container according to claim 3, wherein each of the one or more contact lens chambers is formed in each case by a chamber base which can be closed with an associated chamber lid, the chamber base being formed by a depression in the surface of the cartridge, and the respective chamber lid being arranged on the outer housing.

6. Container according to claim 5, wherein each chamber lid is designed as a deformable membrane which cooperates with an indicating element which can be viewed from outside the outer housing.

7. Container according to claim 1, wherein the liquid reservoir is refillable.

8. Container according to claim 1, further comprising an energy storage device for storing electrical energy.

9. Container according to claim 5, further comprising an energy storage device for electrical energy; and
wherein a number of vibration elements associated with the chamber lid(s) are connected to the energy storage device.

10. Container according to claim 8, wherein an electrical display unit is connected to the energy storage device.

11. Container according to claim 8, wherein an activation module for exciting electroluminescent properties of contact lenses is connected to the energy storage device.
